(19) United States
(12) Patent Application Publication
(10) Pub. No.: US 2004/0116663 A1
(43) Pub. Date: Jun. 17, 2004

(54) Method for refolding of proteins

(76) Inventors: Soren Buus, Bronshoj (DK); Henrik Ferre, Vanlose (DK); Emmanuel Ruffet, Kobenhavn V. (DK)

Correspondence Address: MERCHANT & GOULD PC, P.O. BOX 2903, MINNEAPOLIS, MN, 55402-0903, US

(21) Appl. No.: 10/466,194

(22) PCT Filed: Jan. 16, 2002
(86) PCT No.: PCT/DK02/00032

(30) Foreign Application Priority Data
Jan. 16, 2001 (DK) PA200100070

(51) Int. Cl. C07K 14/47

(52) U.S. Cl. 530/350

(57) ABSTRACT

Method of refolding proteins from a suspension comprising the proteins in a predominantly misfolded form. The method involves adding of a denaturant to the suspension comprising the misfolded proteins to obtain the proteins in a substantially unfolded form. The suspension comprising the unfolded proteins is diluted so as to obtain a mixture where at least part of the proteins are refolded. Subsequently, the refolded proteins can be separated.

### FIELD OF INVENTION

The present Invention relates in its broadest aspect to the field of protein biochemistry in particular refolding of proteins from suspensions containing proteins in an essentially misfolded and thus inactive form. More specifically, there is provided a novel method where misfolded proteins are unfolded and subsequently proper refolded using a diluting step, which allows the refolding conditions to be accurately defined and maintained. This is especially obtained by controlling and maintaining the concentration of protein to be refolded. This secures proper refolding of the proteins before recovery and purification. The method also provides for capture of the refolded protein allowing the refolding buffer to be recycled. Thus, by using this method the total yield of refolded protein can be increased when compared with current methods.

### TECHNICAL BACKGROUND AND PRIOR ART

It is well known in the art that misfolded proteins must be denatured and subsequently refolded in order to regain their native structure and functionality.

The most abundant source of misfolded proteins is found in the production of recombinant proteins from heterologous DNA sequences inserted into host organisms or cell lines. Such heterologous proteins produced by transformant cells are frequently not biologically active because they do not fold into the proper tertiary structure when produced. The heterologous proteins tend to form aggregates, which are recognised within the cell as “inclusion bodies”. Additionally, multimeric complexes and aggregates may occur as the result of inter- and intra-molecular interactions. A special case of the latter are the interactions caused by the formation of covalent intermolecular disulfide bonds. Such complexes may even occur under storage of highly purified proteins in a solution where the proteins tend to form aggregates with themselves.

As a result, protein aggregates needs to be solubilised or denatured and unfolded before proper refolding can be initiated. Existing methods for in vitro solubilising or denaturation and refolding of proteins from misfolded proteins, protein aggregates and inclusion bodies into their native structure are described in the art. These methods include general processes and processes developed for particular proteins. All current process s describe utilisation of strong chaotrophic agents for denaturing of the protein. In vitro refolding is subsequently initiated by reducing the concentration of denaturant leading the solvated, unfolded molecule to refold. This occurs through stage(s) with intermediate conformation(s) eventually leading to a native conformation. During such a refolding process, however, it is unavoidable that misfolded complexes and aggregates are generated as by-products. These occur when the protein to be refolded interacts inappropriately with itself (intramolecular interactions) or with other proteins (intermolecular interactions). The net outcome of a refolding process depends upon the number of productive rearrangements, which lead to native molecules, vs. the number of non-productive rearrangements, which lead to misfolded complexes and aggregations. Obviously, intramolecular interactions are concentration independent, whereas, intermolecular interactions are concentration dependent. The higher the protein concentration, the higher the risk of intermolecular misfolding, and vice versa. Thus, an in vitro refolding process will suffer the least number of intermolecular interactions, and thereby misfoldings, if the refolding protein can be diluted to the extent that any given refolding molecule will be highly unlikely to meet any other refolding molecule, or any already misfolded molecule.

Albeit, refolding can be very fast, it is not instantaneous. A classical refolding leads to a heterogeneous mixture of protein species (some en route towards appropriate folding, some towards misfolding, some already correctly folded, some already misfolded or aggregated). A fact that further complicates the refolding processes as described in the art is that the exact composition of the refolding mixture changes during the refolding process. Thus, the composition of the protein species, as described above, in the refolding mixture will change from the first molecule starts refolding in virtual isolation (best chance of successful rearrangement) to the last molecule refolds in the presence of already folded or misfolded proteins (worst chance of successful rearrangement).

Technically, refolding is accomplished by dialysis or by dilution. Dialysis is a very slow and cumbersome process and there is no simple technical solution as how to control and maintain refolding conditions. Dilution can obviously be done much faster, however, even this method fails to control and maintain refolding conditions. Classical dilution is a batch-wise procedure whereby one (or several) volume(s) containing the unfolded protein suspension is added to a larger volume of renaturing buffer. The speed by which these two volumes are mixed is at best predetermined and controlled. However, the resulting protein species and concentrations are not controlled. Thus, there are several problems associated with the current technology: a) the refolding conditions are poorly controlled and b) the refolding protein concentration and composition is changing leading to decreased yield of active molecules and increased generation of misfolded by-products. Finally, c) large volumes of potentially non-clear solutions are generated, thus, stressing down-stream processing. Accordingly, there are no description in the art disclosing methods for refolding of proteins were the refolding process is controlled, the volume of renaturing buffer reduced by optionally recycling the buffer and with a high yield of protein monomers.

EP 0212960 discloses a method for purifying and solubilising a protein that is produced as insoluble, impure inclusion bodies in a transformant microorganism. The method comprises extraction of the inclusion body by SDS to solubilise the protein. Subsequently, the protein is treated with urea, to obtain the protein in unfolded form, and isolated by chromatography. Finally, the obtained protein solution is dialysed allowing the protein to refold.

WO 00102901 discloses a method for producing renatured biologically active protein from a solution containing denatured protein. The method comprising the steps of obtaining a mixture of protein, adding a chaotrophic agent and subsequently increasing the pressure between 0,25 kbar to about 3,5 kbar. After a time of incubation the pressure is reduced and the protein refolded. The method is a batch refolding, the high pressure allows for reduced use of refolding buffer.

There are in the scientific literature examples of protein refolding that have been done in batch procedures and then loaded onto an EBA (BioSeparations 6:265, 1996). The authors fail to acknowledge the importance of keeping the concentration of protein to be refolded low as they experience protein precipitation even under their preferred refolding conditions (water). Further, they state that “the volume of water was kept to a minimum to keep the final volume down” (i.e. the concentration of protein to be refolded was kept high). Thus, the reference teaches away the importance of diluting the protein in order to secure optimal refolding.

Another example of using EBA as a capture step after refolding is reported as a meeting abstract (Lee, Third International Conference on Expanded Bed Adsorption, EBA 2000, 14-17 May). The method describes loading unfolded molecule on an EBA under fully denaturing conditions. Subsequently, the denaturant are washed away to effect refolding of the proteins on the EBA.

Neither of the disclosed references refers to the importance of controlling the refolding conditions.

The present invention provides for a novel dilution process which allows the refolding conditions, and the concentration of protein to be refolded, to be accurately controlled throughout the refolding process. Accurate control of the dilution step is accomplished by the use of a mixing chamber, which through several inlets allows the unfolded protein, the refolding buffer and any additive to be mixed at any predetermined concentrations. The output of this dilution process may be lead directly into a capture step such as EBA, which can handle excessive volumes of non-clear suspensions. This is accomplished by attaching the mixing chamber outlet with the capture step inlet. By optionally varying the length, and thereby the volume, of said connecting tubing, the time afforded to refolding in solution can be adjusted. The net result of this invention is therefore that the exact refolding conditions can be controlled and maintained so that every single refolding molecule from the very first to the very last will be exposed to identical refolding conditions.

### SUMMARY OF THE INVENTION

Accordingly, the present invention pertains to a method for obtaining from a first suspension comprising a protein in a predominantly misfolded form, a preparation of said protein where at least a part of the protein is in a refolded form, the method comprising the steps of (i) adding a denaturant to the first suspension comprising the misfolded protein to obtain a second suspension comprising the protein in a substantially unfolded form, (ii) diluting the second suspension comprising the unfolded protein to obtain a mixture where at least part of the protein is refolded, and (iii) subjecting said mixture to a separating process permitting separation of refolded protein.

### DETAILED DESCRIPTION OF THE INVENTION

The objective of the present invention is to provide a method for obtaining, from a suspension comprising a protein in a predominantly misfolded form, a preparation of the protein where at least a part of the protein is refolded. Particularly, it is an objective of the present invention to provide a method which makes it possible to initiate refolding of proteins by diluting the suspension comprising proteins in a predominantly unfolded form before recovery of the proteins. The diluting procedure secures proper folding of the protein and a subsequent separation step using Expanded Bed Absorption (EBA) chromatography permits the refolded protein to be purified in a high yield.

Hence, a significant feature of the method of the present invention is that it makes use of this diluting step before subjecting the refolded protein to a separation step. It is important that the unfolded proteins are diluted to an extent which prevents neighbouring proteins to engage in intermolecular interaction, which would result in aggregates and misfolding of the proteins.

According to the method of the present invention, control of the diluting step allows for proper refolding of the unfolded proteins and a reduced use of refolding buffer as the buffer may be recycled, according to particular embodiment of the invention. Additionally, the method of the invention is characterised as being performed continuously and on-line whereby time-consumption and costs can be reduced and the yield of refolded protein increased compared to known methods. By combining refolding with a separation step in an on-line continuous fashion, the method of the present invention ensures a fast and efficient removal of contaminants from the protein of interest, thereby reducing inadvertent modifications, such as proteolysis.

As described above it is appreciated that the method of the invention can be performed on-line. In the present context, the term “on-line” refer to an ongoing process substantially without breaks and include, but are not limited to, terms such as “continuous”, “non-stop”, “permanent”, “constant”, “unbroken” and “uninterrupted”. An on-line process may further be characterised as a process that allows monitoring and/or manipulation while in operation.

The term “continuous” refer to a process, which can proceed uninterrupted and indefinitely. It is contemplated that the method according to the invention may be performed continuously and non-continuously as well.

The proteins to be refolded according to the method of the present invention include all natural and synthetically produced proteins. The proteins to be refolded may be biologically functional. By the term “functional” is meant a protein which is capable of performing at least one of the functions attributed to said protein at least to a substantially degree e.g. as assessed by an in vitro assay. It is contemplated that the proteins to be refolded can be comprised in any solution as a plurality of proteins, at least one of them being in the need of refolding.

The present invention is exemplified with reference to the beta-2 microglobulin component of the MHC class I proteins. However, as mentioned above, all proteins may be refolded according to the method of the invention. Such proteins include e.g. a protein of the immunoglobulin superfamily i.e. a protein selected from the group consisting of antibodies, immunoglobulin variable (V) regions, immunoglobulin constant (C) regions, immunoglobulin light chains, immunoglobulin heavy chains, CD1, CD2, CD3, Class I and Class II histocompatibility molecules, β2microglobulin (β2m), lymphocyte function associated antigen-3 (LFA-3) and FcγRIII, CD7, CD8, Thy-1 and Tp44 (CD28), T cell receptor, CD4, polyimmunoglobulin receptor, neuronal cell adhesion molecule (NCAM), myelin associated glycoprotein (MAG), P myelin protein, carcinoembryonic antigen (CEA), platelet derived growth factor receptor (PDGFR), colony stimulating factor-1 receptor, αβ-glycoprotein, ICAM (intercellular adhesion molecule), platelet and interleukins.

More specifically, a protein for refolding according to the method of the invention is a protein selected from the group consisting of proteins comprising a heavy chain, a heavy chain combined with a β₂m, a functional mature MHC class I protein, and a MHC class II protein selected from the group consisting of an α/β dimer and an α/β dimer with a peptide.

In a specific embodiment of the method of the invention the protein to be refolded is a MHC class I protein including MHC and human MHC. The produced MHC protein to be refolded may be obtained as a peptide free MHC protein.

Thus, the origin of the protein to be refolded may be eukaryotic as well as prokaryotic. The eukaryotic proteins include proteins derived from a vertebrate species selected from the group consisting of humans, a murine species, a rat species, a porcine species, a bovine species and an avian species.

Furthermore, the protein to be refolded may be derived from recombinant protein expression in transformed host organisms or cell lines. Useful prokaryotic cells for expression can be selected from Gram negative and Gram positive bacteria. Examples of useful Gram negative expression cells include Enterobacteriaceae species such as e.g. Escherichia spp. Salmonella spp. and Serratia spp; Pseudomonadanaceae species such as Pseudomonas spp., and examples of Gram positive bacteria that can be used in the invention include Bacillus spp., Streptomyces spp and lactic acid bacterial species. Suitable eukaryotic cells for expression can be selected from fungal cells including yeast cells, mammalian cells including human cells and insect cells.

It is known in the art that the tertiary structure of a native protein will usually have only one, or very few, conformations or forms, where it will be at least partially soluble, and be capable of performing its function. A native protein can—spontaneously or due to extrinsic factors such as denaturants, pH, temperature etc—loose more or less of its native structure and thereby its function. It can partially unfold into intermediary conformations (such as the “molten globular state”), which either refold and regain activity or further unfold and loose activity. Upon further unfolding (denaturing) the molecule can obtain a state of random coil or complete unfolding. Hydrophobic surfaces will frequently be exposed during such unfolding leading to inappropriate associations within the molecule itself or with other molecules resulting in complexes ranging from soluble homo- and/or hetero-multimers to insoluble aggregates. Frequently, these complexes are of an irreversible nature and can only be dissociated and solvated under strongly denaturing conditions.

Recombinant protein expression often results in the formation of insoluble aggregates and it is to be understood that the misfolded protein according to the method of the invention can be part of any structure selected from the group consisting of inclusion bodies, aggregates, insoluble complexes, intermolecular complexes and intramolecular complexes. The tendency to form insoluble aggregates does not correlate with protein characteristics such as the size of the expressed polypeptide, the use of fusion constructs, the subunit structure, or the relative hydrophobicity of the recombinant protein. Overproduction by itself is frequently sufficient to induce the formation of inactive aggregates. Studies of recombinant protein expression in e.g. Escherichia coil have shown that inclusion body formation is a very common phenomenon.

Accordingly, the “first suspension” of the method of the present invention refers to a medium comprising the protein in a predominantly unfolded form. It is to be understood that the proteins in the first suspension may be in an insoluble (e.g. aggregate or complexes as defined above) as well as on a soluble form.

In order to solubilise and unfold the misfolded protein, the first suspension according to step (i) of the method of the present invention is treated with a substance that can keep the proteins in a substantially unfolded form including random coils. Such media include denaturants typically selected from the group consisting of organic solvents such as ethanol and propanol; chaotrophic agents such as urea, guanidin hydrochloride, thiocyanate; detergents such as sodium dodecyl sulfate (SDS), cetyltrimethylammonium bromide (CTAB) or deoxychoiate; salts such as KSCN or LiBr. The concentration of the chaotrophic agent such as urea may be in the range of 3-9 M such as 5-7 M including about 6 M.

The denaturing step of the present invention (step (i)) may be performed under non-reducing conditions i.e. without altering the redox state. Alternatively, the denaturing step may be performed under reducing conditions. Examples of useful reductants include compounds selected from the group consisting of dithlothreitol, dithioerytritol, gluthathione, cysteine, cystamine and 2-mercaptoethanol.

It may be beneficial to add, during the denaturing step, an agent which inhibits proteolysis. Useful proteolysis inhibitors are described in the art and include compounds selected from the group consisting of cysteine, aspartic acid, serine, metallo proteinase inhibitors such as N-ethyl-maleimide, pepstatin, phenyl methyl sulphonic flouride (PMSF) and EDTA, respectively, and of ATP dependent proteolysis inhibitors such as sodium ortho vanadate.

Furthermore, the unfolded protein comprised in the second solution may be purified by a separation process before subjected to the diluting step. Useful methods for separation are described in the art and may be selected from the group consisting of gel size filtration, ion exchange, hydrophobic interaction, reversed phase, expanded bed absorption, immobilised metal ion affinity chromatography, or other methods know to the person skilled in the art.

The unfolded proteins or at least a part of the unfolded protein may be reduced to break disulfide bonds before the dilution step.

The fully or at least partially solvated and unfolded protein can then be subjected to a dilution step were refolding of the unfolded protein is initiated. By this step a “mixture” result. In a specific embodiment of the method of the invention the dilution step is carried out in a mixing device. It is preferred that the device comprises a) a mixing chamber, b) at least two fluid inlets c) means for accurate control and maintenance of the refolding conditions, and d) at least one fluid outlet for the resulting mixture.

According to the method of the invention the protein to be refolded is diluted in order to allow at least part of the unfolded protein to refold. Before diluting the second suspension, it may be preferred to include a separation step permitting at least part of the unfolded proteins to be isolated. Separation and isolation techniques as described in the art can be used in the present invention. The concentration of protein in the diluting step may be adjusted by the described separation or isolation techniques or by adjusting the initial concentration of protein in the first suspension or by adjusting the flow of the second suspension in the diluting step.

In a specific embodiment of the method of the invention, the diluting step, where the refolding of the unfolded protein is initiated, is carried out in a “mixing device”. It is contemplated that such a device may include any device that allows for diluting according to step (ii) of the method. It is to be understood that the dilution of the first suspension may be controlled by a range of methods, such methods are described in the art and will allow a person of skill in the art to select suitable means for diluting the second suspension.

As described above, methods for refolding of unfolded proteins are described in the art and refolding is typically performed by dilution or dialysis. Upon removal of the denaturing or chaotrophic agent, the protein is exposed to intermediate denaturing concentrations. This protein refolding is a delicate process were folding intermediates are very susceptible to aggregation. Suitable refolding buffers are characterised as fluids allowing the protein to refold. Such buffers are described in the art and include TrisHCl buffer and EDTA. It may be preferred to make a buffer system by including a suitable additive to the buffer system and selecting the proper pH and ionic strength of the buffer system. A buffer system for refolding of the protein in question may easily be designed by the person skilled in the art.

Upon in vitro refolding, as described hereinbefore, misfolding as well as aggregation competes with the correct folding pathway. Thus, aggregation predominates upon refolding above a limiting concentration of protein. Because of the predominant aggregation at high protein concentrations, refolding must be performed at an extremely high dilution.

Therefore, it is a very useful feature of the method according to the present invention that the second solution comprising the unfolded protein is accurately diluted with a renaturing or refolding fluid (buffer) in a mixing device as defined above. Such mixing allows the concentration of refolding protein as well as the concentration of denaturant or of any other agent to be controlled and maintained throughout the refolding procedure. In particular it allows the refolding protein to be kept at concentrations below the critical limit whereby misfolded complexes and aggregations can be reduced or even avoided. The specific dilution of a particular protein to be refolded is dependent on a variety of conditions. However, in accordance with the method of the invention the concentration of protein to be refolded in the refolding buffer may be less than 1 mg/ml such as less than 300 μg/ml, such as less than 100 μg/ml, including less than 30 μg/ml, 10 μg/ml, 3 μg/ml, 1 μg/ml, such as less than 300 ng/ml, including less than 100 ng/ml, 30 ng/ml, 10 ng/ml, or even less than 3 ng/ml.

Furthermore, the diluting step of the method as described leads to at least 5%, 10%, 15%, 20%, 30%, 40%, 50%, 60%, 70%, 80%, 85%, 90%, 95% or even 98% of the unfolded protein being obtainable in a refolded form. Further, as illustrated hereinafter the method of the invention captures protein, which has maintained the biologically activity.

According to the method of the invention, the term “refolding conditions” are used interchangeably with the term “mixing conditions” and the terms refer to all extrinsic as well as intrinsic parameters which can be adjusted during the diluting step. These parameters can be controlled directly as well as indirectly. It is appreciated that the above conditions are controlled in order to ensure proper refolding of the protein in question. Conditions which may influence the refolding of unfolded proteins are described in the art and include physical parameters such as e.g. volume, flow of reactants and buffers, temperature and pressure; chemical parameters including pH, ionic strength, reduction potential, oxidation potential, detergents, protease inhibitors and ATPase inhibitors and enzymatic parameters including heat-shock proteins, oxidating or reducting enzymes and disulfide isomerases.

In the diluting step, it may be advantageous to add an agent which inhibits proteolysis. Examples of useful agents are listed above and it is contemplated that the agent may be added directly to the second suspension before diluting or added during mixing with the diluting liquid.

Additionally, it may be advantageous to adjust the redox potential of the mixture optionally, by using a reductant and oxidant. Useful redox pairs may be selected from the group consisting of reduced glutathione (GSH)/oxidized glutathione (GSSG); cystamine/cysteamine; reduced dithiothreitol (DTTred)/oxidized dithiothreitol (DTTox) or other redox pairs known to the person skilled in the art.

Furthermore, the refolding of unfolded proteins comprised in the second suspension may be facilitated by the addition of auxiliary additives to the diluted solution. Such additives may include but are not limited to compounds selected from the group consisting of Tris, L-arginine, detergent, surfactant and organic solvents.

Additionally, adjusting the inlet flow rates may control the ratio between the second suspension and the diluent. Furthermore, the flow through the mixing device may be controlled and maintained by adjusting outlet flow rate and the flow rate between the inlets and outlet.

In order to minimise the use of fluid e.g. refolding buffer/renaturing buffer during the refolding process, the fluid can in accordance with the invention be recycled after recovery of the refolded proteins.

In a final step (iii) of the method of the invention the second suspension is subjected to a separating process. Separation as well as purification techniques which entraps the refolded protein are described in the art and can easily work with the method as described herein. Useful techniques can be selected from the group consisting of dialysis, filtration, dia-filtration, tangential flow-filtration, gel-filtration, extraction (two-phase extraction), precipitation, centrifugation and chromatographic methods.

In a preferred embodiment expanded bed absorption (EBA) chromatography is used for separating and purifying the refolded protein. This technique which also includes the fluid bed absorption chromatography are well known in the art and the methods have the advantages that impure solutions can be added directly to the column without any problem of clotting. Furthermore, the method can operate with large volumes.

In the method according to the present invention the recovery of refolded protein is at least 5%, 10%, 15%, 20%, 30%, 40%, 50%, 60%, 70%, 80%, 85%, 90% or 95%. The total yield may be at least 10 mg refolded protein, including at least 100 mg refolded protein, such as at least 1 g refolded protein, 10 g refolded protein, 100 g refolded protein, including at least 1 kg refolded protein, 10 kg refolded protein, 100 kg refolded protein, or even under large scale refolding at least 1 t of refolded protein.

In a further aspect the present invention relates to a system for obtaining, from a first suspension comprising a protein in a predominantly misfolded form, a preparation of said protein where substantially all the protein is in a refolded form, the system comprising the means for:

(i) adding a denaturant to the first suspension comprising the misfolded protein to obtain a second suspension comprising the protein in a substantially unfolded form,

(ii) diluting the second suspension comprising the unfolded protein to obtain a mixture where at least part of the protein is refolded, and

(iii) subjecting said mixture to a separating process permitting separation of refolded protein.

It should be understood that the system according to this further aspect of the invention may be adapted to carry out any function or step described above in connection with the method according to the fst aspect of the invention.

### DESCRIPTION OF DRAWINGS

The invention is further illustrated in the following non-limiting examples and in the figures, where:

FIG. 1 is an exemplary chart illustrating the set-up during refolding of proteins. The following part are presented in the figure: 1) Expanded bed chromatography column, 2) refolding buffer/renaturing buffer reservoir, 3) reservoir for first solution comprising misfolded or unfolded proteins 4) mixing device, 5) pump circulating a flow of the mixture through the system 6) recycling of the buffer to the refolding buffer reservoir. It is noted that the pump (5) may be positioned anywhere in the recycling pathway. Additionally, a pump (not shown in FIG. 1.) is positioned between (3) and (4) controlling the flow rate of the first solution into the mixing chamber.

FIG. 2 shows the elution profile from the Streamline DEAE column which have been capturing human H6FXa-b2m refolded according to the method of the invention, and

FIG. 3 shows the production of refolded monomeric human H6FXa-b2m (diamonds denoted I) and the concomitant production of misfolded H6FXa-b2m (squares denoted II) as a function of the concentration of refolding H6FXa-b2m.

### EXAMPLE 1

### Methods and Materials

Urea, phenylmethylsulfonyl flouride (PMSF), isopropyl-b-D thiogalactidase (IPTG), bicinchoninic acid solution (BCA) and tris[hydroxymethyl]aminomethane (tris) were purchased from Sigma. Streamline 25, Sephadex G50 and Q-sepharose fast flow anion-exchange material were purchased from Pharmacia, Sweden

### Production Human Beta-2 Microglobulin

Recombinant human beta-2 microglobulin inserted into the pT7H6 vector and expressed in BL21 (DE3) has been described previously (Pedersen et al., 1995). This vector contains a hexahistine (H6) tag followed by a Factor Xa (FXa) cleavage site fused in front of the mature human beta-2 microglobulin. Recombinant BL21 (DE3) cells were plated on a Luria-Bertani (LB)-Ampicillin plate and grown overnight at 37 C. One clone was picked sterile from an over night culture were inoculated and grown in 200 ml LB medium in 200 μg/ml ampicillin in a thermoshaker. Initially the temperature was set to 37C, but as soon as visible growth was observed the temperature is reduced to 25-30C to reduce growth. Shortly after a fermentor culture is seeded with an inoculum corresponding to 10 ml at OD600=1.

A 2 L fermentor (Labfors) was prepared with sterile media containing 8 g K2HPO4, 2 g KH2PO4, 2 g NH4Cl, 4.8 g K2SO4, 264 mg CaCl2.2H2O, 20 ml trace solution, 200 primatone, 6 g yeast extract, 91 g 87% glycerol and 0.3 ml Antifoam 289 (Sigma). The fermentor was autoclaved and allowed to cool off. It was then started on the Labfors fermentor stand at slow agitation (100 rpm) and hooked up to a sterile supply of the acid phosphoric acid and the base NH4OH for the purpose of regulating the pH of the fermentor to be exactly 7. The oxygen electrode was polarized and calibrated after 6 hours. The temperature was set at 25° C. 200 μg/ml ampicillin was added to the fermentor.

10 ml of an OD600=1 inoculum was transferred from the shaker culture to the fermentor. The temperature was set at 25-28° C. and the pH to 7. The fermentor was connected to compressed air. The agitation was set at 800-1200 rpm (regulated by oxygen at threshold 20%). The fermentor was grown at the conditions overnight (approx. 16 hours). The following morning a 1 ml sample was withdrawn, diluted and the OD600 measured. The temperature was then raised to 37 C and the air source was changed to 100% oxygen. The pH was maintained at 7. Samples were withdrawn every hour, diluted and OD600measured. The cells were In exponential growth and the culture was Induced at OD600=25. This was performed by the addition of 1 mM isopropyl-b-d-thiogalacsidase (IPTG). Another batch of 200 μg/ml ampicillin was added to the fermentor and the temperature was raised to 42 C (this temperature increased protein production rate additionally and increased inclusion body formation). The pH was maintained at 7 and the oxygen supply maintained at 100% oxygen. Samples were withdrawn every hour for the purpose of performing an SDS-PAGE analysis of the expression of the induced recombinant protein. Production was continued for 3-5 hours.

### Purification of Inclusion Bodies

The fermentor was harvested and the bacteria recovered by centrifugation. The bacterial pellet was weighted. The cell pellet was resuspended in 300 ml/100 g pellet of a lysis buffer (50 mM Tris HCl, pH 8, 1 mM EDTA, 100 mM NaCl) in a 1 L beaker using a food-processor for 2 min to assure proper resuspension. Then 230 mM PMSF was added and then 80 mg lysozyme/100 g pellet. The cells were incubated at room temperature with occasional stirring until the liquid became thick and slimy (about 20 min). The lysed cells were transferred to a 5 L beaker and the volume was increased to 2 L. Another batch of 10 PMSF was added to 230 mM, and 2.7 g Deoxycholate (DOC) was added. The beaker was incubated at 37° C. and stirred several times until the liquid became highly viscous (about 20 min). The solution was then food processed for 1 min to reduce viscosity. 13.5 mg DNAse I and 20 mg RNAse was added together with 2 mM MgCl₂. The solution was incubated at room temperature until the viscosity disappeared. This solution was food-processed for 3 min and centrifuged at 15000 g for 10 min at 4° C. The pellet was washed twice in PBS with 0.1% DOC and 0.5% NonIdet-P40 (NP40) followed by three washes in lysis buffer (without EDTA). Resuspension after each wash was performed with a food-processor. After the final wash the purified inclusion bodies were obtained. The recombinant protein purity at this stage was >80%.

### Dissolving the Inclusion Bodies (Unfolding the Misfolded Protein)

The inclusion bodies were redissolved in “urea-buffer” (8 M urea containing 50 mM Tris, 500 mM NaCl, pH 8) (freshly made and purified with a mixed bed resin). A volume of 200 ml 8M urea was used to dissolve the inclusion bodies corresponding to 100 g cell pellet. A food processor (1 min) was used to assure proper solvation of the inclusion bodies. The solution was incubated for 20 at 4 C and centrifuged at 15000 g for 15 min at 4 C. The supernatant was transferred to a fresh tube and stored at −20 C.

### Analysis

Samples withdrawn during the fermentor induction, and throughout the inclusion body purification and dissolving were analysed by SDS-PAGE. One-dimensional mini-slap SDS-polyacrylamid gel electrophoresis (PAGE) was performed in homogeneous polyacrylamide gels (15%). Samples were boiled in Laemmli sample buffer with or without 50 mM DTT prior to SDS-PAGE analysis. Proteins were stained with Coomassle Blue R-250. Peptide binding to recombinant MHC class I molecules was performed as previously described.

### Purification of Recombinant Protein While Denatured and Before Refolding

The H6FXa tagged denatured protein in “urea-buffer” was subjected to Nickel (NiNTA) column chromatography. 20-30 ml with 0.5 g recombinant protein was absorbed to a 100 ml NiNTA column, which was washed extensively in “urea-buffer” and subsequently eluted with “urea-imidazol-buffer” (urea-buffer containing 500 mM Imidazol, pH 8).

### Refolding and Recovery

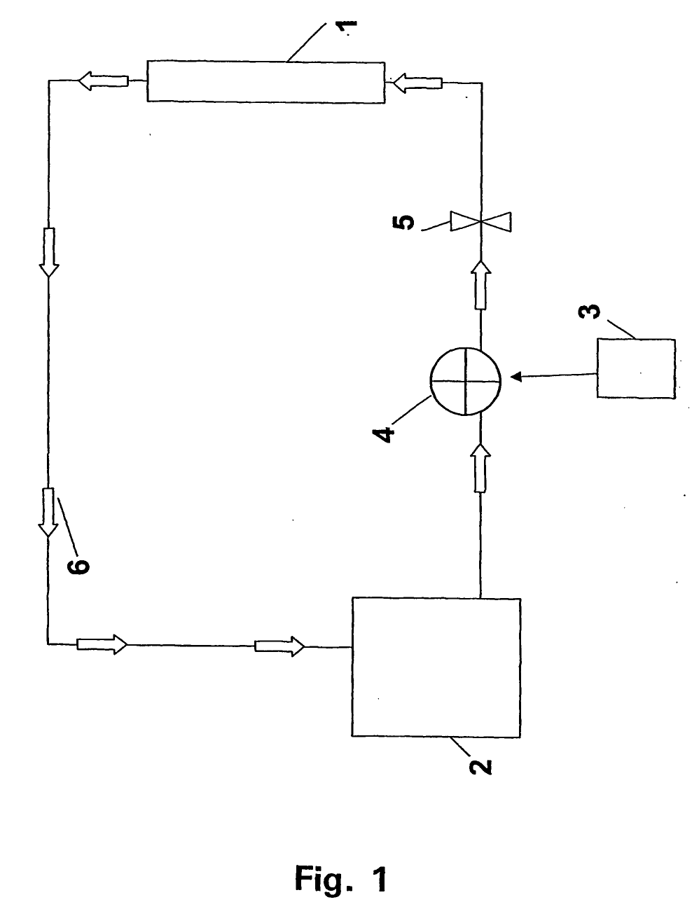

A mixing chamber was purchased from Microlab (Århus, Denmark). It had a mixing volume of 8 ml, had two inlets and one outlet. It was equipped with an impeller, which could be driven by a magnetic stirrer. One of the inlets was fed from a 10 L reservoir with “refolding buffer” (20 mM Tris pH 8). The other inlet was fed from a small reservoir with the urea-buffer solvated recombinant protein and its flow rate was controlled by a Pharmacia P1 pump. The outlet was connected to a Watson-Marlow pump set at 1500-2500 ml/hour to a Streamline 25 equipped with Streamlilne-DEAE gel (about 100 ml packed mode, 250 ml expanded mode, total capacity for human b2m about 0.5 g). This set-up is exemplified in FIG. 1. The exact concentration of refolding protein could be controlled and maintained by adjusting the outlet flow rate of the refolding buffer and the inlet flow rate of the denatured protein. The refolding was performed at room temperature. When all the recombinant protein had been subjected to the refolding treatment, the Streamline column was washed with 10 column volumes refolding buffer. The flow was stopped and the column allowed to settle. The Streamline was attached to a chromatography system (Äkta FPLC, Pharmacia). The column was packed (in a reverse flow direction compared to the expanded bed situation). The column was eluted in 4 column volumes “eluting-buffer 0.5” (refolding buffer containing 0.5 M NaCl) followed by two column volumes “eluting buffer 1” (refolding buffer containing 1M NaCl) followed by 2 column volumes “urea-elution buffer” (8 M urea, 20 mM Tris, 1 M NaCl, pH 8) followed by 2 column volumes “urea-2-ME-elution buffer” ( 8 M urea, 10 mM 2-ME, 20 mM Tris, 1 M NaCl, pH 8). The elution profile was monitored at OD280. The entire elution profile was fractionated and the individual fractions were subjected to SDS-PAGE.

### Purification and Cleavage of Refolded H6FXa-Human-b2m

Fractions containing monomeric forms of human H6FXa-b2m were pooled. Typically, 200 ml containing 200 mg refolded H6FXa-b2m was concentrated to about 45 ml on a Amicon YM3 ultrafiltration device driven by a nitrogen pressure source.

The concentrated human H6FXa-b2m solution was adjusted to 50 mM Tris, 100 mM NaCl, 1 mM CaCl₂, 0.1 mM NiSO₄ and 1 mg/L Factor Xa (FXa) (Protein Engineering, \dot{A}rhus) was added and the mixture was incubated at room temperature for 2 days. Cleavage was monitored by SDS-PAGE.

### Purification Concentration and Validation of Native Human b2m

The FXa releases intact native human b2m from the refolded human H6FXa-b2m protein. The resulting buffer will contain a mixture of undigested human H6FXa-b2m, native human b2m, and the released H6FXa-tag peptide. To remove the hexa-histidine containing tag peptide and any undigested human H6FXa-b2m, this mixture were re-applied to a 65 ml NiNTA column and the effluent containing the refolded human b2m protein was concentrated to 20 ml on an Amicon YM3 filtration device as described above and applied to a 2500 ml Sephadex G50M gel filtration column, which was eluted in 50 mM Tris, 200 mM NaCl pH 8, and fractionated. The fractions were analysed by SDS-PAGE and the fractions containing the refolded human native b2m monomers were pooled and concentrated (Amicon YM3) to a final concentration between 5 and 10 mg/ml.

The ability to support peptide binding to MHC class I—the most stringent test of the functionality of the refolded b2m molecules—was performed as described in PA 1998 01155.

### Results

Recombinant BL21(DE3) were grown in a 2 L Labfors fermentor, induced with 1 mM IPTG at a cell density about 25 and incubated for 3 hours at 42° C. The electrophoretic mobility of boiled and reduced samples with and without IPTG were analyzed in 15% SDS-PAGE gels. Yields of recombinant b2m were estimated to be about 1-2 g/L culture (or up to 4 g per production). To isolate the inclusion bodies, the cells were ruptured by lysozyme and detergent mediated lysis releasing DNA/RNA as well as inclusion bodies. To remove the former, DNAse, RNAse and MgCl were added. After clearance of the solution (20-30 min. at 22° C.), it was centrifuged to pellet the inclusion bodies. Pellet was washed extensively and finally resolubilized in 8 M urea and stored at −20° C. The yield at this stage was typically 1-2 g/L cell culture at a purity of >80%.

The partially purified proteins from inclusion bodies were in some cases further fractionated, using Nickel column chromatography.

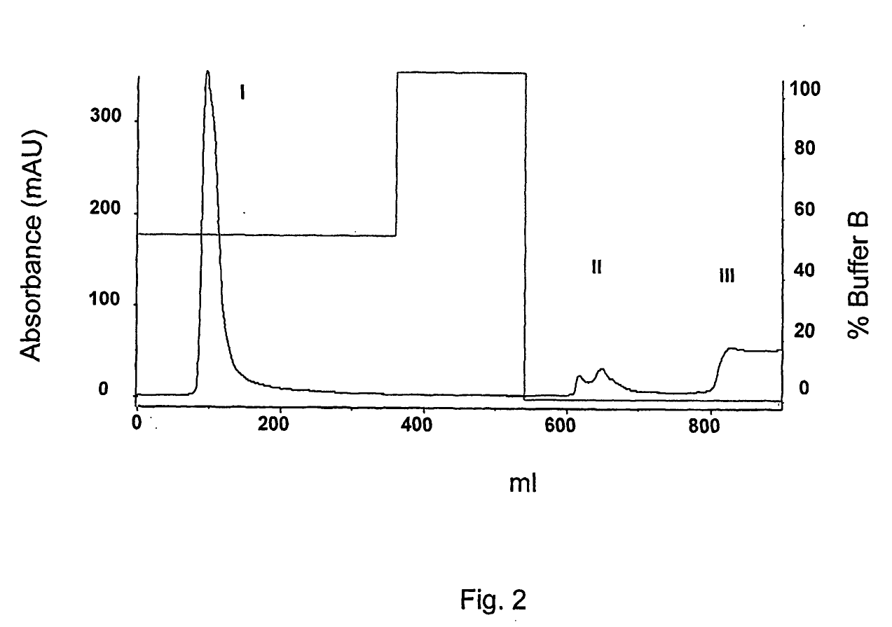

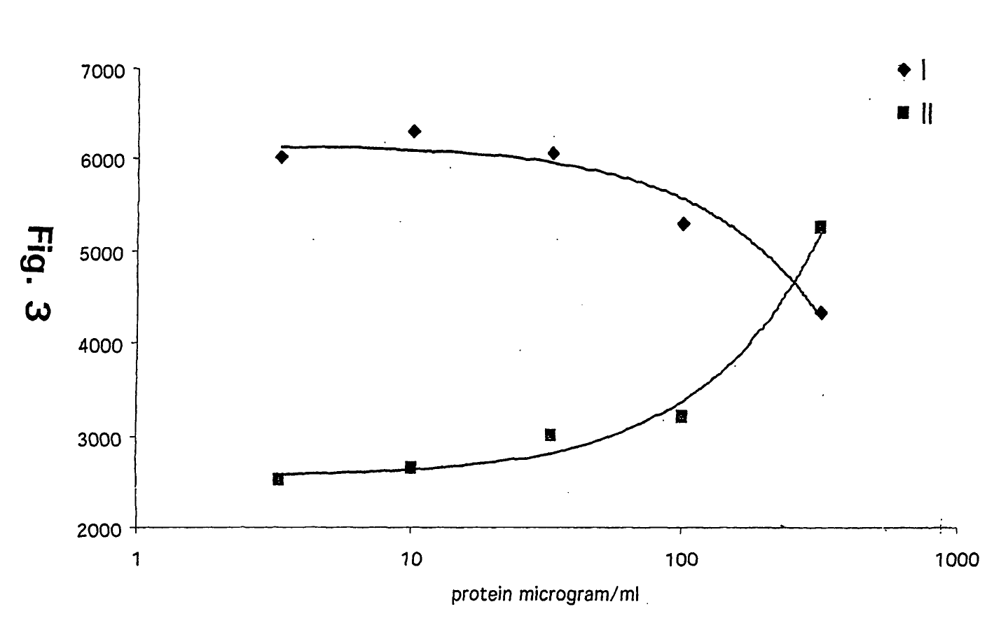

Refolding was initiated by diluting the denatured preparation into refolding buffer in the mixing device described above. For the analytical runs, 20 mg samples of the denatured protein preparation was serially diluted in 8M Urea. The following concentrations was obtained 15 mg/ml, 5 mg/ml, 1.67 mg/ml, 0.5 mg/ml and 0.167 mg/ml. The denatured solution inlet flow rate controlled by the Pharmacia P1 pump was set to 0.5 m/min while the outlet refoldingbuffer flow rate was set at 25 ml/min. By this design, the protein concentration varies from experiment to experiment but is kept constant throughout any given experiment; Thus, it took only 2.7 min to deliver the 20 mg at the high protein concentration and some 4 hours to deliver it at the low protein concentration. The urea is diluted to the same concentration throughout the whole series of experiments. The analytical experiments were done with crude 8M urea extract of the inclusion bodies and also with 8M urea extracted and NiNTA purified preparations. The elution profile from the Streamline shows a) that washing the column in 1 M NaCl elutes a single large symmetric peak, which SDS-PAGE (+/− reduction) analysis shows predominantly b2m monomer (and little dimer), b) that washing the column in 1 M NaCl+8 M Urea elutes a broad less defined peak, which by SDS-PAGE (+/− reduction) contains mostly multimeric versions of the b2m itself, and c) that washing the column in 1 M NaCl+8 M Urea+10 mM 2-ME elutes another broad less defined peak, which by SDS-PAGE contains b2m+ contaminants (these must have been di-sulfide linked prior to the reduction) (FIG. 2.). The results show that the higher the concentration of refolding protein the more misfolded complexes are generated and the less monomer is generated; and that reducing the concentration reduces misfolding and increases correct folding (FIG. 3.). The result also show that the Streamline easily handles the clearly visible aggregates (most prominently seen at high concentrations), that it easily handles the excessive volumes associated with “low protein concentration folding”, and that it easily captures the refolded protein. It also shows significant purification of the captured protein as it appears that only the monomer and a few dimers can be eluted under mild conditions, whereas larger complexes and interactions with contaminants are retained on the column. Thus, using a crude extract input one gets about 25% recovery of monomer after refolding and Streamline capture and elution. Using a NiNTA extract input one gets about 50% recovery of monomer after refolding and Streamline capture and elution.

The above refolding procedure can easily be scaled-up simply by prolonging the time of the refolding process. Our Streamline configuration could purify 500 mg b2m per run. Approximate 400 mg crude 8 M Urea extract, or NiNTA purified protein was refolded at a concentration of refolding protein of 10 μg/ml i.e. it required the use of 40 L refolding buffer. The elution profiles and SDS-PAGE analysis showed good scalability. Thus, the recovery and purities were largely similar to those obtained in the analytical experiments.

The refolded protein was concentrated by ultrafilttration, digested with FXa, released H6FXa tag absorbed on a NiNTA column and the intact native b2m purified by Sephadex G50M gelfiltration and concentration by ultrafiltration. The resulting b2m was more than 95% pure by SDS-PAGE.

The functionality of the recombinant b2m was ascertained in a peptide-MHC class I interactions assay where b2m is absolutely needed to support peptide binding. Support was obtained already at concentrations between 3 and 10 nM b2m and full support was obtained around 1 mM. These values are slightly better than those observed for b2m preparations generated by other methods (chromatofocusing of b2m obtained from urine, or reiterative folding of recombinant, or simple dilution).

The results show that this refolding procedure

Easily handles non-clear solutions

Easily handles large flow rates

Easily handles large volumes

Enables refolding at very low protein concentrations leading to improved folding

Captures the refolded protein

Purifies the captured protein

Enables continuous, on-line folding

Is scalable

Is fast

1. An online method for obtaining, from a first suspension comprising a protein in a predominantly misfolded form, a preparation of said protein where at least a part of the protein is in a refolded form, the method comprising the steps of
(i) adding a denaturant to the first suspension comprising the misfolded protein to obtain a second suspension comprising the protein in a substantially unfolded form,
(ii) diluting the second suspension comprising the unfolded protein to obtain a mixture where at least part of the protein is refolded, and
(iii) subjecting said mixture to a separating process permitting separation of refolded protein.

2. A method according to claim 1 wherein the step of diluting is performed in a mixing device, comprising
a: a mixing chamber,
b: at least two fluid inlets
c: means for accurately controlling and maintaining the refolding conditions, and
d: at least one fluid outlet for the resulting mixture.

3. A method according to claim 1 or 2 wherein a plurality of proteins, at least one of these being in the need of refolding, are present during the refolding.

4. A method according to any of claims 1-3 wherein the process is continuous.

5. A method according to any of claims 1-4 wherein at least part of the unfolded protein is purified from the second suspension by a separation process before diluting the second suspension.

6. A method according to any of claims 1-5 wherein at least part of the unfolded protein is subjected to reducing conditions to break disulfide bonds before diluting the second suspension.

7. A method according to any of claims 1-6 wherein the concentration of the protein to be refolded is controlled.

8. A method according to any of claims 1-7 wherein the refolding conditions of step (ii) are controlled with respect to physical parameters selected from the group consisting of volume, flow of reactants and buffers, temperature and pressure.

9. A method according to any of claims 1-8 wherein the refolding conditions of step (ii) are controlled with respect to chemical parameters selected from the group consisting of pH, ionic strength, reduction potential, oxidation potential, detergents, protease inhibitors and ATPase inhibitors.

10. A method according to any of claims 1-9 wherein the refolding conditions are controlled with respect to enzymatic parameters selected from the group consisting of heat-shock proteins, oxidizing or reducing enzymes and disulfide isomerases.

11. A method according to any of claims 1-10 wherein the refolding conditions are controlled by adjusting the outlet flow rate.

12. A method according to any of claims 1-11 wherein the renaturing buffer is recycled after separation of the refolded protein.

13. A method according to any of claims 1-12 wherein the protein is an immunoglobulin superfamily protein selected from the group consisting of antibodies, immunoglobulin variable (V) regions, immunoglobulin constant (C) regions, immunoglobulin light chains, immunoglobulin heavy chains, CD1, CD2, CD3, Class I and Class II histocompatibility molecules, β₂microglobulin (β₂m), lymphocyte function associated antigen-3(LFA-3) and FcγRIII, CD7, CD8, Thy-1, Tp44 (CD28), T cell receptor, CD4, polyimmunoglobulin receptor, neuronal cell adhesion molecule (NCAM), myelin associated glycoprotein (MAG), P myelin protein, carcinoembryonic antigen (CEA), platelet derived growth factor receptor (PDGFR), colony stimulating factor-1 receptor, αβ-glycoprotein, ICAM (intercellular adhesion molecule), platelet and interleukins.

14. A method according to any of claims 1-13 wherein the protein is derived from a vertebrate species selected from the group consisting of humans, a murine species, a rat species, a porcine species, a bovine species and an avian species.

15. A method according to any of claims 1-14 wherein the protein is a MHC.

16. A method according to claim 15 wherein the MHC protein is a human MHC.

17. A method according to claim 15 or 16 wherein the MHC protein is a MHC class I protein selected from the group consisting of a heavy chain, a heavy chain combined with a β₂m, a functional mature MHC class I protein and a MHC class II protein selected from the group consisting of an α/β dimer and an α/β dimer with a peptide.

18. A method according to any of claims 16-17 wherein the produced MHC protein is obtained as a peptide free MHC protein.

19. A method according to any of claims 1-18 wherein at least 5%, 10%, 15%, 20%, 30%, 40%, 50%, 60%, 70%, 80%, 85%, 90%, 95%, or 98% of the produced protein is obtained in a refolded form.

20. A method according to any of claims 1-19 wherein the misfolded protein can be part of any structure selected from the group consisting of inclusion bodies, aggregates, intermolecular complexes, intramolecular complexes and random coils.

21. A method according to any of claims 1-20 wherein misfolded protein is unfolded in step (i) by a medium that can keep the protein essentially unfolded.

22. A method according to claim 21 wherein the medium is a denaturant selected from the group consisting of organic solvents, chaotrophic agents, detergents and salts.

23. A method according to claim 22 wherein the denaturant is urae at a concentration in the range of 3-9 M such as 5-7 M including about 6 M.

24. A method according to any of claims 1-23 wherein step (i) is performed under reducing conditions.

25. A method according to any of claim 1-23 wherein step (i) is performed under non-reducing conditions without altering the redox state.

26. A method according to claim 24 wherein the reductant is selected from the group consisting of dithiothreitol, dithioerytritol, gluthathione, cysteine, cystamine and 2-mercaptoethanol.

27. A proces according to any of claims 1-26 wherein an agent which inhibit proteolysis is added in the denaturing step (i).

28. A method according to any of claims 1-27 wherein an agent which inhibit proteolysis is added in the dilution step (ii)

29. A method according to claims 27-28 wherein the proteolysis inhibitor(s) is selected from the group consisting of N-ethyl-maleimide, pepstatin, phenyl methyl sulphonic flouride (PMSF) and EDTA, respectively, and of ATP dependent proteolysis inhibitors such as sodium ortho vanadate.

30. A method according to claims 1-29 wherein the redox potential in the refolding step (ii) is adjusted using a mixture of reductant and oxidant

31. A method according to claim 30 wherein the redox pair is selected from the group consisting of reduced glutathione (GSH)/oxidized glutathione (GSSG); cystamine/cysteamine.

32. A method according to any of claims 1-31 wherein the refolding is facilitated by the addition of auxiliary additives to the mixture chamber in step (ii).

33. A method according to claims 32 wherein additives are selected from the group consisting of Tris, L-arginine, detergents, surfactants and organic solvents.

34. A method according to any of claims 2-33 wherein the refolding conditions of step (ii) is accurately controlled and maintained by controlling the flow rate the inlets and outlets, of the volume of the mixing chamber and efficient mixturing.

35. A method according to any of claims 1-34 wherein the concentration of protein to be refolded in the refolding buffer and thus second suspension may be less than 1 mg/ml including less than 300 μg/ml, such as less than 100 μg/ml, including less than 30 μg/ml, 10 μg/ml, 3 μg/ml, 1 μg/ml, such as less than 300 ng/ml, including less than 100 ng/ml, 30 ng/ml, 10 ng/ml, or even less than 3 ng/ml.

36. A method according to any of claim 1-35 wherein the separation process in step (iii) is selected from the group consisting of dialysis, filtration, dia-filtration, tangential flow filtration, gel-filtration, extraction (two-phase extraction), precipitation, centrifugation and chromatography.

37. A method according to claim 37 wherein the chromatography is expanded bed absorption chromatography.

38. A method according to claim 37 wherein the filtration method is tangential flow-filtration.

39. A method according to any of claims 1-38 wherein the recovery of refolded protein is at least 5%, 10%, 15%, 20%, 30%, 40%, 50%, 60%, 70%, 80%, 85%, 90%, 95% or 98%

40. A method according to any of claims 1 to 39 wherein the yield of the process is at least 10 mg refolded protein, 100 mg refolded protein, 1 g refolded protein, 10 g refolded protein, 100 g refolded protein, 0.1 kg refolded protein, 10 kg refolded protein, 100 kg refolded protein or 1 t refolded protein.
